(19) United States
(12) Patent Application Publication
(10) Pub. No.: US 2017/0114371 A1
(43) Pub. Date: Apr. 27, 2017

(54) Process For Producing Ethanol From Starch Using A GH5 Xylanase

(71) Applicant: Novozymes A/S, Bagsvaerd (DK)

(72) Inventors: Sven Pedersen, Gentofte (DK); Jens Magnus Eklöf, Copenhagen (DK)

(73) Assignee: Novozymes A/S, Bagsvaerd (DK)

(21) Appl. No.: 15/318,439

(22) PCT Filed: Jul. 9, 2015
(86) PCT No.: PCT/EP2015/065752

§ 371 Date: Dec. 13, 2016

(30) Foreign Application Priority Data
Jul. 10, 2014 (EP) 14176468.8
Jul. 10, 2014 (EP) 14176473.8
Oct. 20, 2014 (EP) 14189543.3

(51) Int. Cl. C12P 7/10; C12P 19/02; C12P 19/14

(52) U.S. Cl. CPC C12P 7/10; C12P 19/14; C12P 19/02

(57) ABSTRACT

The present invention relates to a process for producing fermentation products from starch-containing material, wherein a GH5 xylanase is present during saccharification.

### REFERENCE TO A SEQUENCE LISTING

This application contains a Sequence Listing in computer readable form, which is incorporated herein by reference.

### BACKGROUND OF THE INVENTION

Field of the Invention

The present invention relates to a process for producing a fermentation product from starch-containing material.

Description of the Related Art

Xylans are hemicelluloses found in all land plants (Popper and Tuohy, Plant Physiology, 2010, 153:373-383). They are especially abundant in secondary cell walls and xylem cells. In grasses, with type II cell walls, glucurono arabinoxylans are the main hemicellulose and are present as soluble or insoluble dietary fiber in many grass based food and feed products.

Plant xylans have a 13-1,4-linked xylopyranose backbone that can be substituted at the O2 or O3 position with arabinose, glucuronic acid and acetic acid in a species and tissue specific manner. The starch-rich seeds of the Panicoideae with economically important species such as corn and sorghum have special types of highly substituted xylans in their cell walls. Compared to wheat flour, wherein over 60% of the xylosyl units in the arabinoxylan backbone are unsubstituted. In corn kernel xylan, the corresponding percentage of unsubstituted backbone xylosyls is 20-30%, and in sorghum it is 35-40% (Huismann et al. Carbohydrate Polymers, 2000, 42:269-279). Furthermore, in corn and sorghum the xylan side chains can be longer than a single arabinose or glucuronic acid substitution which is common in other xylans. This added side chain complexity is often due to L- and D-galactose and D-xylose sugars bound to the side chain arabinose or glucuronic acid. About every tenth arabinose in corn kernel xylan is also esterified with a ferulic acid and about every fourth xylose carries an acetylation (Agger et al. J. Agric. Food Chem, 2010, 58:6141-6148). All of these factors combined make the highly substituted xylans in corn and sorghum resistant to degradation by traditional xylanases.

The known enzymes responsible for the hydrolysis of the xylan backbone are classified into enzyme families based on sequence similarity (www.cazy.org). The enzymes with mainly endo-xylanase activity have previously been described in Glycoside hydrolase family (GH) 5, 8, 10, 11 and 30. The enzymes within a family share some characteristics such as 3D fold and they usually share the same reaction mechanism. Some GH families have narrow or mono-specific substrate specificities while other families have broad substrate specificities. Commercially available GH10 and GH11 xylanases are often used to break down the xylose backbone of arabinoxylan. However, such xylanases are sensitive to side chain steric hindrance and whilst they are effective at degrading arabinoxylan from wheat, they are not very effective on the xylan found in the seeds of Panicoideae species, such as corn or sorghum. Corn is used around the world in e.g., animal feed and for producing bioethanol, and thus there is a need to discover new polypeptides having xylanase activity that are capable of breaking down the highly branched xylan backbone in the cell wall in order to release more starch which are trapped inside the cell wall.

### SUMMARY OF THE INVENTION

According to the invention several xylanases belonging to the GH5 family have been shown to be effective in hydrolyzing highly branched xylan backbones and thus these GH5 xylanases can be advantageously applied in starch to ethanol processes. The present invention therefore relates to a process for producing fermentation products from starch-containing material comprising the steps of:
a) liquefying starch-containing material using an alpha-amylase;
b) saccharifying the liquefied material using a glucoamylase;
c) fermenting using a fermenting organism, wherein saccharification is carried out in the presence of a GH5 xylanase.

### BRIEF DESCRIPTION OF THE FIGURES

FIG. 1 shows the effect on ethanol yield by adding a GH5 xylanase during saccharification. A liquefied mash was split into three parts and simultaneous saccharification and fermentation performed. Fermentation was followed by weight loss measured twice a day and by HPLC at the end of fermentation.

### DEFINITIONS

Arabinoxylan-containing material: The term “Arabinoxylan-containing material” means any material containing arabinoxylan. Arabinoxylan is a hemicellulose found in both the primary and secondary cell walls of plants, including woods and cereal grains, consisting of copolymers of two pentose sugars, arabinose and xylose. The arabinoxylan chain contains a large number of 1,4-linked xylose units. Many xylose units are substituted with 2-, 3- or 2,3-substituted arabinose residues.

Examples of arabinoxylan-containing material are forage, roughage, seeds and grains (either whole or prepared by crushing, milling, etc. from e.g. corn, oats, rye, barley, wheat), trees or hard woods (such as poplar, willow, eucalyptus, palm, maple, birch), bamboo, herbaceous and/or woody energy crops, agricultural food and feed crops, animal feed products, cassava peels, cocoa pods, sugar cane, sugar beet, locust bean pulp, vegetable or fruit pomaces, wood waste, bark, shavings, sawdust, wood pulp, pulping liquor, waste paper, cardboard, construction and demolition wood waste, industrial or municipal waste water solids or sludge, manure, by-product from brewing and/or fermentation processes, wet distillers grain, dried distillers grain, spent grain, vinasse and bagasse.

Forage as defined herein also includes roughage. Forage is fresh plant material such as hay and silage from forage plants, grass and other forage plants, grass and other forage plants, seaweed, sprouted grains and legumes, or any combination thereof. Examples of forage plants are Alfalfa (Lucerne), birdsfoot trefoil, brassica (e.g. kale, rapeseed (canola), rutabaga (swede), turnip), clover (e.g. alsike clover, red clover, subterranean clover, white clover), grass (e.g. Bermuda grass, brome, false oat grass, fescue, heath grass, meadow grasses, miscanthus, orchard grass, ryegrass, switchgrass, Timothy-grass), corn (maize), hemp, millet, barley, oats, rye, sorghum, soybeans and wheat and vegetables such as beets. Crops suitable for ensilage are the ordinary grasses, clovers, alfalfa, vetches, oats, rye and maize. Forage further includes crop residues from grain production (such as corn stover; straw from wheat, barley, oat, rye and other grains); residues from vegetables like beet tops; residues from oilseed production like stems and leaves form soy beans, rapeseed and other legumes; and fractions from the refining of grains for animal or human consumption or from fuel production or other industries.

Roughage is generally dry plant material with high levels of fiber, such as fiber, bran, husks from seeds and grains and crop residues (such as stover, copra, straw, chaff, sugar beet waste).

Preferred sources of arabinoxylan-containing materials are forage, roughage, seeds and grains, sugar cane, sugar beet and wood pulp.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Highly branched xylan: The term “highly branched xylan” means that more than 50% of xylosyl units in the arabinoxylan backbone are substituted. This is preferably calculated from linkage analysis as performed in Huismann et al. Carbohydrate Polymers, 2000, 42:269-279.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).

Mature polypeptide: The term “mature polypeptide” means a polypeptide in its final form following translation and any post-translational modifications, such as N-terminal processing, C-terminal truncation, glycosylation, phosphorylation, etc. In one aspect, the mature polypeptide is amino acids 37 to 573 of SEQ ID NO: 2. Amino acids 1 to 27 of SEQ ID NO: 2 are a signal peptide. Amino acids 28 to 36 of SEQ ID NO: 2 are a his-tag. In another aspect the mature polypeptide is amino acids 36 to 582 of SEQ ID NO: 4. Amino acids 1 to 27 of SEQ ID NO: 4 are a signal peptide. Amino acids 28 to 35 of SEQ ID NO: 4 are a his-tag. In another aspect the mature polypeptide is amino acids 36 to 633 of SEQ ID NO: 6. Amino acids 1 to 27 of SEQ ID NO: 6 are a signal peptide. Amino acids 28 to 35 of SEQ ID NO: 6 are a his-tag. In another aspect the mature polypeptide is amino acids 1 to 620 of SEQ ID NO: 7. In another aspect the mature polypeptide is amino acids 1 to 865 of SEQ ID NO: 8.

It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide.

Mature polypeptide coding sequence: The term “mature polypeptide coding sequence” means a polynucleotide that encodes a mature polypeptide having xylanase activity. In one aspect, the mature polypeptide coding sequence is nucleotides 109 to 1719 of SEQ ID NO: 1. Nucleotides 1 to 81 of SEQ ID NO: 1 encode a signal peptide. Nucleotides 82 to 108 of SEQ ID NO: 1 encode a his-tag. In one aspect, the mature polypeptide coding sequence is nucleotides 106 to 1746 of SEQ ID NO: 3. Nucleotides 1 to 81 of SEQ ID NO: 3 encode a signal peptide. Nucleotides 82 to 105 of SEQ ID NO: 3 encode a his-tag. In one aspect, the mature polypeptide coding sequence is nucleotides 106 to 1899 of SEQ ID NO: 5. Nucleotides 1 to 81 of SEQ ID NO: 5 encode a signal peptide. Nucleotides 82 to 105 of SEQ ID NO: 5 encode a his-tag.

Oligosaccharide composition: The term “oligosaccharide composition” means olig-and poly saccharides but does not include mono- and disaccharides (DP1 and DP2).

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Percentage solubilised xylose: The term “percentage solubilised xylose” means the amount of xylose measured in the supernatant after incubation with an enzyme compared to the total amount of xylose present in the substrate before the incubation with the enzyme. The percentage solubilised xylose from defatted destarched maize (DFDSM) may be calculated as described in example 3 herein.

Sequence Identity: The relatedness between two amino acid sequences or between two nucleotide sequences is described by the parameter “sequence identity”.

For purposes of the present invention, the degree of sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 3.0.0 or later. Version 6.1.0 was used. The optional parameters used are gap open penalty of 10, gap extension penalty of 0.5, and the EBLOSUM62 (EMBOSS version of BLOSUM62) substitution matrix. The output of Needle labelled “longest identity” (obtained using the—nobrief option) is used as the percent identity and is calculated as follows:

(Identical Residues×100)/(Length of Alignment−Total Number of Gaps in Alignment)

For purposes of the present invention, the degree of sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. Version 6.1.0 was used. The optional parameters used are gap open penalty of 10, gap extension penalty of 0.5, and the EDNAFULL (EMBOSS version of NCBI NUC4.4) substitution matrix. The output of Needle labelled “longest identity” (obtained using the—nobrief option) is used as the percent identity and is calculated as follows:

(Identical Deoxyribonucleotides×100)/(Length of Alignment−Total Number of Gaps in Alignment)

Xylanase: The term “xylanase” means a 1,4-beta-D-xylan-xylohydrolase (E.C. 3.2.1.8) that catalyses the endohydrolysis of 1,4-beta-D-xylosidic linkages in xylans. Xylanase activity can be determined with 0.2% AZCL-arabinoxylan as substrate in 0.01% TRITON® X-100 and 200 mM sodium phosphate pH 6 at 37° C. One unit of xylanase activity is defined as 1.0 μmole of azurine produced per minute at 37° C., pH 6 from 0.2% AZCL-arabinoxylan as substrate in 200 mM sodium phosphate pH 6.

GH5 xylanase: The xylanases described in this invention belongs to GH5, a GH family with a wide array of substrate specificities. The relationship between sequences within GH5 has been clarified by defining subfamilies of related sequences (Aspeborg et al. BMC Evolutionary Biology, 2012, 12:186). Subdividing a GH family into subfamilies has significantly improved the predictive power for substrate specificity, not only for GH5 (Lombard et al. Nucleic Acids Res, 2014, 42:D490-D495). Two of the subfamilies of GH5, GH5_21 and GH5_34, have been described as xylanases acting on arabinoxylan. Interestingly, these two xylanase subfamilies are not closely related and are the result of convergent evolution. During the course of this work, members of subfamily GH5_35, a subfamily of previously unknown function, has been found to have xylanase activity.

### DETAILED DESCRIPTION OF THE INVENTION

The inventors have found that certain xylanases from glycoside hydrolase family 5 (herein referred to as GH5) are surprisingly good at degrading the xylose backbone of sterically hindered arabinoxylan, thereby releasing increased amounts of xylose. In particular GH5 xylanases belonging to subfamilies GH5_21, GH5_34 and GH5_35 all have the capacity to access and degrade highly branched xylans (meaning that more than 50% of xylosyl units in the arabinoxylan backbone are substituted) that are resistant to xylanase degradation by xylanases of other GH families. Increased degradation, and thereby increased xylose release, increase accessibility of starch trapped in the xylose structure. Thus the present invention relates to process for producing fermentation products from starch-containing material comprising the steps of:
a) liquefying starch-containing material using an alpha-amylase;
b) saccharifying the liquefied material using a glucoamylase;
c) fermenting using a fermenting organism, wherein saccharification is carried out in the presence of a GH5 xylanase.

### GH5 Xylanases

The GH5 xylanases applied in the process of the invention are in one embodiment selected from the group consisting of subfamilies 21, 34, or 35.

In one specific embodiment the subfamily 21 GH5 xylanase is selected from the xylanase shown as amino acids 36 to 633 of SEQ ID NO: 6, or a GH5 xylanase having at least 75%, at least 80%, at least 85%, at least 90%, at least 95%, at least 96%, at least 97%, at least 98%, at least 99% identity to amino acids 36 to 633 of SEQ ID NO: 6.

In another specific embodiment the subfamily 34 GH5 xylanase is selected from the xylanase shown as SEQ ID NO: 7, or a GH5 xylanase having at least 75%, at least 80%, at least 85%, at least 90%, at least 95%, at least 96%, at least 97%, at least 98%, at least 99% identity to SEQ ID NO: 7.

In another specific embodiment the subfamily 34 GH5 xylanase is selected from the xylanase shown as SEQ ID NO: 8, or a GH5 xylanase having at least 75%, at least 80%, at least 85%, at least 90%, at least 95%, at least 96%, at least 97%, at least 98%, at least 99% identity to SEQ ID NO: 8.

In another specific embodiment the subfamily 35 GH5 xylanase is selected from the xylanase shown as amino acids 37 to 573 of SEQ ID NO: 2, or a GH5 xylanase having at least 75%, at least 80%, at least 85%, at least 90%, at least 95%, at least 96%, at least 97%, at least 98%, at least 99% identity to amino acids 37 to 573 of SEQ ID NO: 2.

In another specific embodiment the subfamily 35 GH5 xylanase is selected from the xylanase shown as amino acids 36 to 582 of SEQ ID NO: 4, or a GH5 xylanase having at least 75%, at least 80%, at least 85%, at least 90%, at least 95%, at least 96%, at least 97%, at least 98%, at least 99% identity to amino acids 36 to 582 of SEQ ID NO: 4.

Production of Fermentation Products from Starch-Containing Materials

Processes for Producing Fermentation Products from Gelatinized Starch-Containing Material

In this aspect the present invention relates to a process for producing a fermentation product, especially ethanol, from starch-containing material, which process includes a liquefaction step and sequentially or simultaneously performed saccharification and fermentation steps.

The invention relates to processes for producing fermentation products from starch-containing material comprising the steps of:
i) liquefying starch-containing material using an alpha-amylase:
ii) saccharifying the liquefied material using a carbohydrate-source generating enzyme;
iii) fermenting using a fermenting organism, wherein saccharification is carried out in the presence of a GH5 xylanase.

The fermentation product, such as especially ethanol, may optionally be recovered after fermentation, e.g., by distillation. Suitable starch-containing starting materials are listed in the section “Starch-Containing Materials”-section below. In an embodiment the starch-containing materials is corn or what. Contemplated enzymes are listed in the “Enzymes”-section below. The liquefaction is carried out in the presence of an alpha-amylase, preferably a bacterial alpha-amylase, especially Bacillus alpha-amylase, such as a Bacillus stearothermophilus alpha-amylase. The fermenting organism is preferably yeast, preferably a strain of Saccharomyces, especially a strain of Saccharomyces cerevisae. Suitable fermenting organisms are listed in the “Fermenting Organisms”-section above. In a preferred embodiment steps ii) and iii) are carried out sequentially or simultaneously (i.e., as SSF process).

The aqueous slurry may contain from 10-55 wt.- % dry solids, preferably 25-45 wt.- % dry solids, more preferably 30-40 wt.- % dry solids of starch-containing material. The slurry is heated to above the initial gelatinization temperature. Alpha-amylase, preferably bacterial alpha-amylase, may be added to the slurry. In an embodiment the slurry is also jet-cooked to further gelatinize the slurry before being subjected to an alpha-amylase in liquefaction step i).

The temperature during step (i) is above the initial gelatinization temperature, such as between 80-90° C., such as around 85° C.

In an embodiment liquefaction is carried out as a three-step hot slurry process. The slurry is heated to between 60-95° C., preferably between 80-90° C., and alpha-amylase is added to initiate liquefaction (thinning). Then the slurry is jet-cooked at a temperature between 95-140° C., preferably 105-125° C., for 1-15 minutes, preferably for 3-10 minutes, especially around 5 minutes. The slurry is cooled to 60-95° C., preferably 80-90° C., and more alpha-amylase is added to finalize hydrolysis (secondary liquefaction). The liquefaction process is usually carried out at pH 4.5-6.5, in particular at a pH between 5 and 6. Milled and liquefied starch is known as “mash”.

The saccharification in step ii) may be carried out using conditions well known in the art. For instance, a full saccharification process may last up to from about 24 to about 72 hours. In an embodiment a pre-saccharification step is done at 40-90 minutes at a temperature between 30-65° C., typically at about 60° C., followed by complete saccharification during fermentation in a simultaneous saccharification and fermentation step (SSF). Saccharification is typically carried out at temperatures from 30-70° C., such as 55-65° C., typically around 60° C., and at a pH between 4 and 5, normally at about pH 4.5.

The most widely used process in fermentation product production, especially ethanol production, is simultaneous saccharification and fermentation (SSF) process, in which there is no holding stage for the saccharification.

SSF may typically be carried out at a temperature between 25° C. and 40° C., such as between 28° C. and 36° C., such as between 30° C. and 34° C., such as around 32° C., when the fermentation organism is yeast, such as a strain of Saccharomyces cerevisiae, and the desired fermentation product is ethanol. In an embodiment fermentation is ongoing for 6 to 120 hours, in particular 24 to 96 hours.

Other fermentation products may be fermented at conditions and temperatures, well known to the skilled person in the art, suitable for the fermenting organism in question. According to the invention the temperature may be adjusted up or down during fermentation.

In an embodiment a protease is adding during fermentation. Examples of proteases can be found in the “Proteases”-section below.

In a preferred embodiment step (ii) and step (iii) are carried out as a simultaneous saccharification and fermentation process. In such preferred embodiment the process is typically carried at a temperature between 25° C. and 40° C., such as between 28° C. and 36° C., such as between 30° C. and 34° C., such as around 32° C. According to the invention the temperature may be adjusted up or down during fermentation.

In an embodiment simultaneous saccharification and fermentation is carried out so that the sugar level, such as glucose level, is kept at a low level such as below 6 wt.- %, preferably below about 3 wt.- %, preferably below about 2 wt.- %, more preferred below about 1 wt.- %., even more preferred below about 0.5%, or even more preferred 0.25% wt.- %, such as below about 0.1 wt.- %. Such low levels of sugar can be accomplished by simply employing adjusted quantities of enzyme and fermenting organism. A skilled person in the art can easily determine which quantities of enzyme and fermenting organism to use. The employed quantities of enzyme and fermenting organism may also be selected to maintain low concentrations of maltose in the fermentation broth. For instance, the maltose level may be kept below about 0.5 wt.- % or below about 0.2 wt.- %.

The process of the invention may be carried out at a pH in the range between 3 and 7, preferably from pH 3.5 to 6, or more preferably from pH 4 to 5.

In an embodiment a protease is adding during fermentation. Examples of proteases can be found in the “Proteases”-section below.

### Starch-Containing Materials

According to the invention sugars may be derived from starch-containing materials. Any suitable starch-containing starting material, including granular starch, may be used according to the present invention. The starting material is generally selected based on the desired fermentation product. Examples of starch-containing starting materials, suitable for use in a process of present invention, include whole grains, corns, wheat, barley, rye, triticale, milo, sago, cassava, tapioca, sorghum, rice, peas, beans, and sweet potatoes, or mixtures thereof, or cereals, or sugar-containing raw materials, such as molasses, fruit materials, sugar cane or sugar beet, potatoes. Contemplated are both waxy and non-waxy types of corn and barley.

In a particular embodiment the starch containing material comprises maize, corn, wheat, rye, barley, triticale, sorghum, switchgrass, millet, pearl millet, foxtail millet or in a processed form such as milled corn, milled wheat, milled rye, milled barley, milled triticale, milled maize, defatted maize, defatted destarched maize, milled sorghum, milled switchgrass, milled millet, milled foxtail millet, milled pearl millet.

Particularly the starch based material comprises corn, sorghum, wheat, rye, barley, or triticale.

The term “granular starch” means raw uncooked starch, i.e., starch in its natural form found in cereal, tubers or grains. Starch is formed within plant cells as tiny granules insoluble in water. When put in cold water, the starch granules may absorb a small amount of the liquid and swell. At temperatures up to 50° C. to 75° C. the swelling may be reversible. However, with higher temperatures an irreversible swelling called “gelatinization” begins. Granular starch to be processed may in an embodiment be a highly refined starch, preferably at least 90%, at least 95%, at least 97% or at least 99.5% pure, or it may be a more crude starch containing material comprising milled whole grain including non-starch fractions such as germ residues and fibers. The raw material, such as whole grain, is milled in order to open up the structure and allowing for further processing. Two milling processes are preferred according to the invention: wet and dry milling. In dry milling whole kernels are milled and used. Wet milling gives a good separation of germ and meal (starch granules and protein) and is often applied at locations where the starch hydrolyzate is used in production of syrups. Both dry and wet milling is well known in the art of starch processing and is equally contemplated for the process of the invention.

The starch-containing material may be reduced in particle size, preferably by dry or wet milling, in order to expose more surface area. In an embodiment the particle size is between 0.05 to 3.0 mm, preferably 0.1-0.5 mm, or so that at least 30%, preferably at least 50%, more preferably at least 70%, even more preferably at least 90% of the starch-containing material fit through a sieve with a 0.05 to 3.0 mm screen, preferably 0.1-0.5 mm screen.

### Fermenting Organisms

The term “fermenting organism” refers to any organism, including bacterial and fungal organisms, including yeast and filamentous fungi, suitable for producing a desired fermentation product. Especially suitable fermenting organisms according to the invention are able to ferment, i.e., convert sugars, such as glucose, fructose, maltose, xylose, mannose and/or arabinose, directly or indirectly into the desired fermentation product. Examples of fermenting organisms include fungal organisms, such as yeast. Preferred yeast includes strains of the genus Saccharomyces, in particular a strain of Saccharomyces cerevisiae or Saccharomyces uvarum; a strain of Pichia, in particular Pichia stipitis or Pichia pastoris; a strain of the genus Candida, in particular a strain of Candida utilis, Candida arabinofermentans, Candida diddensii, Candida sonorensis, Candida shehatae, Candida tropicalis, or Candida boidinii. Other contemplated yeast includes strains of Hansenula, in particular Hansenula polymorpha or Hansenula anomala; strains of Kluyveromyces, in particular Kluyveromyces marxianus or Kluyveromyces fagilis, and strains of Schizosaccharomyces, in particular Schizosaccharomyces pombe.

In an embodiment the fermenting organism is a C6 sugar (hexose) fermenting organism, such as a strain of, e.g., Saccharomyces cerevisiae.

In connection with especially fermentation of lignocellulose derived materials, C5 sugar (pentose) fermenting organisms are also contemplated. Most C5 sugar fermenting organisms also ferment C6 sugars. Examples of C5 sugar fermenting organisms include strains of Pichia, such as of the species Pichia stipitis. C5 sugar fermenting bacteria are also known. Also some Saccharomyces cerevisae strains ferment C5 (and C6) sugars. Examples are genetically modified strains of Saccharomyces spp that are capable of fermenting C5 sugars include the ones concerned in, e.g., Ho et al., 1998, Applied and Environmental Microbiology, p. 1852-1859 and Karhumaa et al., 2006, Microbial Cell Factories 5:18.

In one embodiment the fermenting organism is added in fermentation so that the viable fermenting organism, such as yeast, count per mL of fermentation medium is in the range from 10⁵ to 10¹², preferably from 10⁷ to 10¹⁰, especially about 5×10⁷.

Commercially available yeast includes, e.g., RED START™ and ETHANOL RED™ yeast (available from Fermentis/Lesaffre, USA), FALI (available from Fleischmann's Yeast, USA), SUPERSTART and THERMOSACC™ fresh yeast (available from Ethanol Technology, WI, USA), BIOFERM AFT and XR (available from NABC—North American Bioproducts Corporation, GA, USA), GERT STRAND (available from Gert Strand AB, Sweden), and FERMIOL (available from DSM Specialties).

The fermenting organism capable of producing a desired fermentation product from fermentable sugars, including glucose, fructose maltose, xylose, mannose, and/or arabinose, is preferably grown under precise conditions at a particular growth rate. When the fermenting organism is introduced into/added to the fermentation medium the inoculated fermenting organism pass through a number of stages. Initially growth does not occur. This period is referred to as the “lag phase” and may be considered a period of adaptation. During the next phase referred to as the “exponential phase” the growth rate gradually increases. After a period of maximum growth the rate ceases and the fermenting organism enters “stationary phase”. After a further period of time the fermenting organism enters the “death phase” where the number of viable cells declines.

### Fermentation Products

The term “fermentation product” means a product produced in a process including a fermentation step using a fermenting organism. Fermentation products contemplated according to the invention include alcohols (e.g., ethanol, methanol, butanol); organic acids (e.g., citric acid, acetic acid, itaconic acid, lactic acid, gluconic acid); ketones (e.g., acetone); amino acids (e.g., glutamic acid); gases (e.g., H₂ and CO₂); antibiotics (e.g., penicillin and tetracycline); enzymes; vitamins (e.g., riboflavin, B₁₂, beta-carotene); and hormones.

In a preferred embodiment the fermentation product is ethanol, e.g., fuel ethanol; drinking ethanol, i.e., potable neutral spirits; or industrial ethanol or products used in the consumable alcohol industry (e.g., beer and wine), dairy industry (e.g., fermented dairy products), leather industry and tobacco industry. Preferred beer types comprise ales, stouts, porters, lagers, bitters, malt liquors, happoushu, high-alcohol beer, low-alcohol beer, low-calorie beer or light beer.

### Fermentation

The sugars derived from plant material used in fermentation in a process of the invention may be derived from starch-containing material and/or lignocellulose-containing material. The fermentation conditions are determined based on, e.g., the kind of plant material, the fermentable sugars, the fermenting organism and/or the desired fermentation product. One skilled in the art can easily determine suitable fermentation conditions. The fermentation may according to the invention be carried out at conventionally used conditions. Preferred fermentation processes are anaerobic processes.

Fermentation of Sugars Derived from Starch-Containing Materials

As mentioned above different kinds of fermenting organisms may be used for fermenting sugars derived from starch-containing material. Fermentations are often carried out using yeast, such as Saccharomyces cerevisae, as the fermenting organism. However, bacteria and filamentous fungi may also be used as fermenting organisms. Some bacteria have higher fermentation temperature optimum than, e.g., Saccharomyces cerevisae. Therefore, fermentations may in such cases be carried out at temperatures as high as 75° C., e.g., between 40-70° C., such as between 50-60° C. However, bacteria with a significantly lower temperature optimum down to around room temperature (around 20° C.) are also known. Examples of suitable fermenting organisms can be found in the “Fermenting Organisms”-section above.

For ethanol production using yeast, the fermentation may in one embodiment go on for 24 to 96 hours, in particular for 36 to 72 hours. In an embodiment the fermentation is carried out at a temperature between 20 to 40° C., preferably 28 to 36° C., in particular around 32° C. In an embodiment the pH is from pH 3 to 6, preferably around pH 4 to 5.

Especially contemplated is simultaneous hydrolysis/saccharification and fermentation (referred to as “SSF”) where there is no separate holding stage for the hydrolysis/saccharification, meaning that the hydrolysing enzyme(s), the fermenting organism(s), and calmodulin protein may be added together. However, it should be understood that the calmodulin protein may also be added separately. When fermentation is performed simultaneous with saccharification (i.e., SSF) the temperature is preferably between 20 to 40° C., preferably 28 to 36° C., in particular around 32° C. when the fermentation organism is a strain of Saccharomyces cerevisiae and the desired fermentation product is ethanol.

Other fermentation products may be fermented at temperatures known to the skilled person in the art to be suitable for the fermenting organism in question.

The process of the invention may be performed as a batch or as a continuous process. The fermentation process of the invention may be conducted in an ultrafiltration system where the retentate is held under recirculation in the presence of solids, water, and the fermenting organism, and where permeate is the desired fermentation product containing liquid. Equally contemplated if the process is conducted in a continuous membrane reactor with ultrafiltration membranes and where the retentate is held under recirculation in presence of solids, water, the fermenting organism and where the permeate is the fermentation product containing liquid.

After fermentation the fermenting organism may be separated from the fermented slurry and recycled.

Fermentations are typically carried out at a pH in the range between 3 and 7, preferably from pH 3.5 to 6, such as around pH 5. Fermentations are typically ongoing for 24-96 hours.

### Methods and Processes According to the Invention

The main aspect according to the present invention relates to processes for hydrolysing starch containing material to sugars and subsequently fermenting the sugars to alcohol, particularly ethanol, wherein a GH5 xylanase is present during the hydrolysis. Different processes and process conditions have been described above.

A particular aspect of the invention relates to a process for producing fermentation products from starch-containing material comprising the steps of:
a) liquefying starch-containing material using an alpha-amylase:
b) saccharifying the liquefied material using a glucoamylase;
c) fermenting using a fermenting organism, wherein saccharification is carried out in the presence of a GH5 xylanase.

In a particular embodiment the starch containing material comprises maize, corn, wheat, rye, barley, triticale, sorghum, switchgrass, millet, pearl millet, foxtail millet or in a processed form such as milled corn, milled wheat, milled rye, milled barley, milled triticale, milled maize, defatted maize, defatted destarched maize, milled sorghum, milled switchgrass, milled millet, milled foxtail millet, milled pearl millet.

Particularly the starch based material comprises corn, sorghum, wheat, rye, barley, or triticale.

The GH5 xylanases are particularly effective on highly branched xylans and thus in one embodiment the starch-containing material comprises highly branched xylan.

The GH5 xylanases are preferably selected from the group consisting of subfamily 21, 34, or 35.

In one specific embodiment the subfamily 21 GH5 xylanase is selected from the xylanase shown as amino acids 36 to 633 of SEQ ID NO: 6, or a GH5 xylanase having at least 75%, at least 80%, at least 85%, at least 90%, at least 95%, at least 96%, at least 97%, at least 98%, at least 99% identity to amino acids 36 to 633 of SEQ ID NO: 6.

In another specific embodiment the subfamily 34 GH5 xylanase is selected from the xylanase shown as SEQ ID NO: 7, or a GH5 xylanase having at least 75%, at least 80%, at least 85%, at least 90%, at least 95%, at least 96%, at least 97%, at least 98%, at least 99% identity to SEQ ID NO: 7.

In another specific embodiment the subfamily 34 GH5 xylanase is selected from the xylanase shown as SEQ ID NO: 8, or a GH5 xylanase having at least 75%, at least 80%, at least 85%, at least 90%, at least 95%, at least 96%, at least 97%, at least 98%, at least 99% identity to SEQ ID NO: 8.

In another specific embodiment the subfamily 35 GH5 xylanase is selected from the xylanase shown as amino acids 37 to 573 of SEQ ID NO: 2, or a GH5 xylanase having at least 75%, at least 80%, at least 85%, at least 90%, at least 95%, at least 96%, at least 97%, at least 98%, at least 99% identity to amino acids 37 to 573 of SEQ ID NO: 2.

In another specific embodiment the subfamily 35 GH5 xylanase is selected from the xylanase shown as amino acids 36 to 582 of SEQ ID NO: 4, or a GH5 xylanase having at least 75%, at least 80%, at least 85%, at least 90%, at least 95%, at least 96%, at least 97%, at least 98%, at least 99% identity to amino acids 36 to 582 of SEQ ID NO: 4.

The fermentation product is in one embodiment an alcohol, particularly ethanol.

Saccharification and fermentation may be performed separately or simultaneously. The fermenting organism is preferably a Saccharomyces sp., more particularly Saccharomyces cerevisiae. Suitable commercial yeast strain available have been described in the section “Fermenting organisms” herein.

The present invention is further described by the following numbered embodiments:
Embodiment 1. A process for producing fermentation products from starch-containing material comprising the steps of:
a) liquefying starch-containing material using an alpha-amylase:
b) saccharifying the liquefied material using a glucoamylase;
c) fermenting using a fermenting organism, wherein saccharification is carried out in the presence of a GH5 xylanase.
Embodiment 2. The process according to embodiments 1, wherein the starch containing material comprises maize, corn, wheat, rye, barley, triticale, sorghum, switchgrass, millet, pearl millet, foxtail millet or in a processed form such as milled corn, milled wheat, milled rye, milled barley, milled triticale, milled maize, defatted maize, defatted destarched maize, milled sorghum, milled switchgrass, milled millet, milled foxtail millet, milled pearl millet.
Embodiment 3. The process according to any of the preceding embodiments, wherein the starch-containing material comprises highly branched xylan.
Embodiment 4. The process according to any of the preceding embodiments, wherein the GH5 xylanase is selected from subfamily 21, 34, or 35.
Embodiment 5. The process according to embodiment 4, wherein the subfamily 21 GH5 xylanase is selected from the xylanases shown as amino acids 36 to 633 of SEQ ID NO: 6, or a GH5 xylanase having at least 75% identity to amino acids 36 to 633 of SEQ ID NO: 6.
Embodiment 6. The process according to embodiment 4, wherein the subfamily 34 GH5 xylanase is selected from the xylanases shown as SEQ ID NO: 7, SEQ ID NO: 8, or a xylanase having at least 75% identity to SEQ ID NO: 7, or SEQ ID NO: 8.
Embodiment 7. The process according to embodiment 4, wherein the subfamily 35 GH5 xylanase is selected from the xylanases shown as amino acids 37 to 573 of SEQ ID NO: 2, amino acids 36 to 582 of SEQ ID NO: 4, or a xylanase having at least 75% identity to amino acids 37 to 573 of SEQ ID NO: 2, or amino acids 36 to 582 of SEQ ID NO: 4.
Embodiment 8. The process according to any of the preceding embodiments, wherein the plant material comprises corn, sorghum, wheat, rye, barley, or triticale.
Embodiment 9. The process according to any of the preceding embodiments, wherein the fermentation product is alcohol, particularly ethanol.
Embodiment 10. The process according to any of the preceding embodiments, wherein the fermenting organism is yeast, particularly Saccharomyces sp., more particularly Saccharomyces cerevisiae.
Embodiment 11. The process according to any of the preceding embodiments, wherein steps b) and c) are performed simultaneously.
Embodiment 12. A use of a GH5 xylanase for producing ethanol from starch containing material.
Embodiment 13. The use according to embodiment 12, wherein the GH5 xylanase is selected from subfamily 21, 34, or 35.
Embodiment 14. The use according to embodiments 12-13, wherein starch containing material comprises corn, sorghum, wheat, rye, barley, or triticale.

The present invention is further described by the following examples that should not be construed as limiting the scope of the invention.

### EXAMPLES

Media and Solutions

Preparation of Destarched Maize (DSM)

107 kg of milled maize (<10 mm) was mixed in a tank with 253 kg of tap water at 53° C. to make a slurry. The temperature of the slurry was 47° C. and the pH 5.9. The pH was adjusted to 6.15 with 1 L of 1 N NaOH and the tank was then heated to 95° C. 1.119 kg of Termamyl® alpha-amylase (Novozymes NS, Bagsvaerd, Denmark) was added at 52° C. and incubated for 80 minutes at 95° C. The pH measured at the end of the incubation was 6.17. Cold tap water was added to the slurry and the slurry was centrifuged and decanted 3 times using a Westfalia decanter CA-225-110 (4950±10 rpm, flow ˜600 l/h) giving 64.5 kg of sludge. The sludge was then collected, frozen and freeze-dried to give 17.1 kg of destarched maize (DSM).

### Preparation of Defatted Destarched Maize (DFDSM)

500 mL acetone was added to 100 gram of destarched maize, prepared as described above. The slurry was stirred for 5 minutes and allowed to settle. The acetone was decanted and the procedure was repeated 2 times. The residue was air dried overnight to give defatted destarched maize (DFDSM) which was stored at room temperature.

### Preparation of Destarched Sorghum

Whole sorghum seeds were milled and sieved and a fraction below 0.5 mm was used for further processing. The sieved fraction was suspended in 25 mM NaOAc pH 5.5 at 20% dry matter and destarched. The destarching involved a first step at 85° C. with 500 ppm Termamyl SC alpha-amylase (Novozymes NS, Bagsvaerd, Denmark) for 20 min followed by an overnight incubation using 250 ppm Attenuzyme Flex (Novozymes NS, Bagsvaerd, Denmark) at 65° C. The slurry was centrifuged and the liquid decanted. After this another destarching was made using by adding MilliQ water and 200 ppm Termamyl SC and 200 ppm Attenuzyme Flex and incubating overnight at 65° C.

The sorghum fiber was separated from the liquid by vacuum filtration through a Whatman F glass fiber filter. The filter cake was then washed several times with excess of water to remove soluble sugars. Finally the destarched sorghum fiber was dried in an oven at 65° C. and the dry fiber milled quickly in a coffee grinder so that the particle size was in general less than 1 mm.

### Preparation of Corn Fiber Gum

### Corn Destarching

107 kg pre-milled corn (<1.0 mm) mixed with 253 kg water at 53° C. The mixture was heated to 95° C. and the pH adjusted to 6.2 with 1 M NaOH. 1.12 kg, Termamyl 120 L (a commercial alpha amylase available from Novozymes NS) was added and the reaction was kept around 90° C. for 3 hrs. After 3 hrs, cold water was added to a total reaction weight of 600 kg. Liquid solid separation was done with a Westfalia decanter, CA-225-110, 4950 rpm, flow 600 L/hour. The solid fiber fraction was re-slurried and separated twice with water to a total weight of 600 kg followed by separation as described above. The final fiber fraction was divided into smaller portions and freeze dried for 3-4 days.

Termamyl 120 L is a Bacillus licheniformis alpha amylase, however, other acid alpha amylases showing good temperature stability may also be used. Examples include, a Bacillus stearothermophilus alpha-amylases, which have a double deletion corresponding to delta(181-182) and further comprise a N193F substitution (also denoted 1181* +G182* +N193F) compared to the wild-type BSG alpha-amylase amino acid sequence set forth in SEQ ID NO: 3 disclosed in WO 99/19467.

### Corn Fiber Gum Extraction

20 g of destarched corn was added to 200 mL boiling MiIIiQ™ gether with 0.8 g of NaOH and 0.8 g of Ca(OH)2. The mixture was kept for 1 hour at 96 C and then centrifuged for 20 min at 6000 g.

The supernatant looked milky and fatty and therefore fats were extracted by shaking with hexane (1 part hexane to 4 parts corn fiber gum). After a resting time the hexane was removed by pipetting.

A 2nd extraction (CFG2) was made by dissolving the pellet in 200 mL 1 M NaOH. The mixture was kept for 1 hour at 96 C and then centrifuged for 20 min at 6000 g and the supernatant adjusted to pH 6 with 4 M HCI.

Both CFG fractions were concentrated using a rotavap and precipitated in EtOH at a final concentration of 90%. The pellet from the 0.1 M NaOH will from now on be called CFG1. Due to the high pH in 1 M NaOH extract (CFG2) this fraction was dialysed on a 2 L measuring cylinder with deionised water with the tap dripping overnight using a 3 kDa dialysis membrane.

Before application trials both CFG1 and CFG2 were centrifuged at 25000 g for 25 min and filtered through a 0.44 pm syringe filter. The dry matter in CFG2 fractions was determined with a Mettler Toledo HR73

### Assays

### Xylose Assay

A xylose standard curve from 0 to 125 μg xylose/mL was prepared from a stock solution of 2.5 mg xylose/mL (prepared by dissolving 0.125 g xylose in 50 mL de-ionised water).

Assay principle. The interconversion of the α- and β-anomeric forms of D-xylose is catalysed by xylose mutarotase (XMR) using the D-xylose assay kit from Megazyme International Ireland. The β-D-xylose is oxidised by NAD+ to D-xylonic acid in the presence of R-xylose dehydrogenase (β-XDH) at pH 7.5. The amount of NADH formed in this reaction is stoichiometric with the amount of D-xylose and is measured by the increase in absorbance at 340 nm.

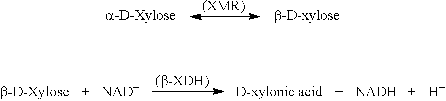

### Example 1

### Cloning of GH5 Xylanases

### Donor Strains

The xylanase from Acetivibrio cellulolyticus CD2 was identified in part of its public genome sequence as originally published under the accession number UniProt: E1KC96 (Lucas S., Copeland A., Lapidus A., Cheng J.-F., Bruce D., Goodwin L.,Pitluck S., Land M. L., Hauser L., Chang Y.-J., Jeffries C., Mouttaki H., He Z., Zhou J., Hemme C. L., Woyke T. J.; “The draft genome of Acetivibrio cellulolyticus CD2.”; Submitted (AUG-2010) to the EMBL/GenBank/DDBJ databases). The xylanase has also been published in Hemme C L, Mouttaki H, Lee Y J, Zhang G, Goodwin L, Lucas S, Copeland A, Lapidus A, Glavina del Rio T, Tice H, et al.: Sequencing of multiple clostridial genomes related to biomass conversion and biofuel production. J Bacteriol 2010, 192(24):6494-6496. Uniprot: El KC96 is SEQ ID NO: 7 and SEQ ID NO:8 are both GH5_34 xylanses.

The xylanase from the strains Paenibacillus sp. 18054 (SEQ ID NO: 3 and 4) and Paenibacillus iffinoisensis (SEQ ID NO: 1 and 2) were identified by shotgun genome sequencing. Both xylanases belong to family GH5_35. The strains were isolated from a thermal sample from New Zealand in 1991. P. illionensis is deposited as DSMZ under accession number DSM16232.

The elephant dung metagenome xylanase (SEQ ID NO: 5 and 6) belongs to GH5_21 family was obtained by deep sequencing of a metagenome extract. The dung of a six years old female Asian elephant (name “Kandy”) living in the zoological garden in Hamburg, Germany was used. The DNA isolation was performed with the QlAamp DNA Stool kit from Qiagen (Hilden, Germany) as described in the manufacturer's protocol.

Genome sequencing, the subsequent assembly of reads and the gene discovery (i.e. annotation of gene functions) is known to the person skilled in the art and the service can be purchased commercially.

### Cloning Examples

Based on the nucleotide sequences mentioned in section/example “Donor strains”, one codon optimized synthetic gene per xylanase was synthesized and purchased commercially. A truncated tri modular version of Uniprot: E1KC96 (A³¹-P⁶⁵⁰) was cloned and is from here on referred to as SEQ ID NO: 7.

The optimization process and the cloning of the purchased synthetic genes were sub-cloned using Clal and Mlul restriction sites into a Bacillus expression vector as described in WO 12/025577. The xylanases were expressed with a Bacillus clausii secretion signal (BcSP; with the following amino acid sequence: MKKPLGKIVASTALLISVAFSSSIASA, originating from the protease AprH of B. clausii). BcSP replaced all native secretion signals respectively in all genes.

Downstream of the BcSP sequence an affinity tag sequence was introduced to ease the purification process (Histag; with the following amino acid sequence: HHHHHHPR for the xylanase from elephant dung metagenome, Paenibacillus sp. 18054 and Acetivibrio cellulolyticus and HQHQHQHPR for the Paenibacillus illinoisensis xylanase, respectively). The gene that was expressed therefore comprised the BcSP sequence followed by the Histag sequence followed by the mature wild type xylanase sequence

The final expression plasmids (BcSP-Histag-xylanase) were individually transformed into a Bacillus subtilis expression host. The xylanase BcSP-fusion genes were integrated by homologous recombination into the Bacillus subtilis host cell genome upon transformation.

The gene construct was expressed under the control of a triple promoter system (as described in WO 99/43835). The gene coding for chloramphenicol acetyltransferase was used as maker (as described in (Diderichsen et al., 1993, Plasmid 30: 312-315). Transformants were selected on LB media agar supplemented with 6 microgram of chloramphenicol per ml. One recombinant Bacillus subtilis clone containing the respective xylanase expression construct was selected and was cultivated on a rotary shaking table in 500 ml baffled Erlenmeyer flasks each containing 100 ml yeast extract-based media. After 3-5 days cultivation time at 30° C. to 37° C., enzyme containing supernatants were harvested by centrifugation and the enzymes were purified by His-tag purification.

### Example 2

### Purification of GH5 Xylanases

All His-tagged enzymes were purified by immobilized metal chromatography (IMAC) using Ni²⁺ as the metal ion on 5 mL HisTrap Excel columns (GE Healthcare Life Sciences). The purification took place at pH 8 and the bound proteins were eluted with imidazole. The purity of the purified enzymes was checked by SDS-PAGE and the concentration of each enzyme determined by Abs 280 nm after a buffer exchange.

### Example 3

### Measurement of Soluble and Insoluble Dietary Fiber in the Substrate Defatted De-Starched Maize (DFDSM) and Correlation to Soluble Xylose Measured After Enzymatic Incubation

400 mg of defatted de-starched maize (DFDSM) was added to NaOAc-buffer (5 mL, pH 5). The mixture was heated to between 90-100° C., then Termamyl 300 DX (100 pL, Novozymes NS, Bagsvaerd, Denmark) was added and the mixture was incubated for 1 hr. The mixture was then cooled and amyloglucosidase from Aspergillus niger (500 μL, catalogue number E-AMGDF, for use in Megazyme Total Starch and Dietary Fiber, Megazyme International Ireland, Wicklow, Ireland) was added and samples were incubated overnight (16 h) at 60° C. The mixture was then cooled and centrifuged at 2500 ×g for 10 min at 5° C. The supernatant was collected and NaOAc-buffer (5 mL, pH 5) was added to the residue and centrifuged at 2500 ×g, 10 min, 5° C. This procedure was repeated twice. The supernatants were then collected, pooled and analysed for soluble NSP as described in A. The residue was analysed for insoluble NSP as described in B.

### A: Soluble NSP, Supernatant

The pooled supernatants were diluted to a fixed volume from which a 5 mL aliquot of supernatant was taken. To this aliquot was added 20.1 mL cold 99.9% ethanol and the mixture was kept on ice for approx. 15 min for precipitation of polymers with a DP>10. After centrifuging at 2500 ×g, 5° C. for 10 min, the supernatant was discarded.

5 mL cold 80% ethanol was added to the pellet and the mixture was kept on ice for approx. 15 min. After centrifuging at 2500 ×g, 5° C. for 10 min, the supernatant was discarded.

Acid hydrolysis of the precipitate was conducted by the addition of MQ water (7.9 mL), myoinositol (0.5 mL, internal standard) and 12M H₂SO₄ (0.3 mL) and autoclaving at 125° C. for 55 minutes.

### B: Insoluble NSP, Residue

The pellet obtained after AMG treatment was hydrolysed by the addition of MQ water (74 mL), myoinositol (10 mL, internal standard) and 12M H₂SO₄ (3 mL) and autoclaving at 125° C. for 55 minutes.

### GLC Analysis

After autoclaving, the samples were reduced with borohydride to produce alditol sugars and these were derivatised by acetylation to become volatile for GLC analyses on an instrument with FID detector (Pettersson et al, (1995) “Total dietary fiber determined as neutral sugar residues, uronic acid residues, and Klason lignin (the Uppsala method), Collaborative study”, J. AOAC Int. 78:1030-1044). The concentration of the soluble or insoluble sugars was determined relative to myo-inositiol.

### Percentage Solubilised Xylose

When DFDSM is incubated with enzyme at 40° C. for 4 hours, the enzyme solubilizes the xylan in the substrate and this solubilized xylan is then hydrolysed further by acid. The xylose released is measured spectrophotometrically using a D-xylose assay kit (Megazyme, catalogue number K-xylose). This xylose (which is actually enzyme solubilized xylan) is then correlated to the amount of total xylose of the substrate measured by GLC as described above.

The DFDSM contains 99% insoluble and 1% soluble xylose, in total 14.81% xylose which represents the concentration of xylose polymer (DP>10) present in the sample (DFDSM) according to the analysis. Based on the release of xylose measured by the Megazyme kit which calculates release based on sample weight, the amount of xylose released can be calculated as follows: e.g. 1% release from 400 mg of sample equals 4 mg of xylose. In 400 mg sample there is 400mg×14.81% xylose, equal to 59.22 mg xylose. The gross xylose (insoluble+soluble) release is that case 4 mg/59.22 mg which represents a release of 6.75 mg xylose, but it should be noted that this value must be corrected for the passive release obtained for the non-enzyme supplemented control. This corrected value is defined herein as the percentage solubilised xylose.

### Example 4

### Hydrolysis of Defatted Destarched Maize (DFDSM) with GH5 Xylanases

Defatted destarched maize (DFDSM, 400 mg) was added to aqueous sodium acetate (0.1 M, 3.9 mL) solution containing calcium chloride (5 mM) at pH 5 and the mixture heated to 40° C. for 30 minutes. 100 μL buffer or enzyme solution was added and the sample was heated at 40° C. for 4 hours. The sample was cooled to 5° C. and centrifuged (4000 rpm, 5° C.) for 10 minutes. 1.7 mL of the sample was transferred to an Eppendorf tube and the enzyme deactivated by heating to 95° C. for 10 minutes. The samples were then frozen until hydrolyzed.

The supernatant was thawed and centrifuged (14000 rpm) for 5 minutes. The supernatant (250 pL) was diluted with Milli-Q water (250 μL) in glass tubes and HCI (1.63 M, 2.0 mL) was added. The reaction was heated to 100° C. for 1 hour then cooled in an ice bath.

Aqueous NaOH solution (1.3 M, 2.5 mL) was added whilst the samples were cooled on ice and the samples were stored at 0-5° C. whilst xylose content was analysed using the xylose assay. The results are presented in tables 2, 3 and 4.

TABLE 2
Xylose release from DFDSM using GH5 xylanase SEQ ID NO: 2

| | | | % solu- | | |
|---|---|---|---|---|---|
| | Conc. | Soluble | bilised | Signifi- | Std. |
| GH5 Xylanase | [mg EP/kg] | xylose (%) | xylose¹ | cance² | Dev. |
| Blank | 0 | 0.039 | 0.3 | C | 0.004 |
| Ronozyme WX | 25 | 0.101 | 0.7 | C | 0.009 |
| (GH11) | | | | | |
| SEQ ID NO: 2 | 10 | 0.903 | 6.2 | B | 0.116 |
| SEQ ID NO: 2 | 25 | 1.295 | 9.0 | A | 0.225 |
| SEQ ID NO: 2 | 50 | 1.200 | 8.2 | A | 0.133 |

¹Percentage solubilised xylose was calculated as described in example 3.
²ABC: Least squared values within a column not sharing a capital letter differ significantly (P < 0.05 all pairs Tukey-Kramer HSD).

Table 2 shows the amount of xylose measured after acid hydrolysis of supernatants (% of dry matter and % solubilized xylose of total xylose) when incubating defatted de-starched maize (DFDSM) with the GH5 xylanase of SEQ ID NO: 2 at different enzyme concentrations compared to blank and the commercial GH11 xylanase Ronoxyme WX.

TABLE 3
Xylose release from DFDSM using GH5 xylanase SEQ ID NO: 4

| | | | % solu- | | |
|---|---|---|---|---|---|
| | Conc. | Soluble | bilised | Signifi- | Std. |
| GH5 Xylanase | [mg EP/kg] | xylose (%) | xylose¹ | cance² | Dev. |
| Blank | 0 | 0.040 | 0.3 | C | 0.001 |
| Ronozyme WX | 25 | 0.101 | 0.7 | C | 0.010 |
| (GH11) | | | | | |
| SEQ ID NO: 4 | 10 | 0.890 | 5.9 | B | 0.018 |
| SEQ ID NO: 4 | 25 | 1.016 | 7.0 | B | 0.078 |
| SEQ ID NO: 4 | 50 | 1.340 | 9.2 | A | 0.280 |

¹Percentage solubilised xylose was calculated as described in example 3.
²ABC: Least squared values within a column not sharing a capital letter differ significantly (P < 0.05 all pairs Tukey-Kramer HSD).

Table 3 shows the amount of xylose measured after acid hydrolysis of supernatants (% of dry matter and % solubilized xylose of total xylose) when incubating defatted de-starched maize (DFDSM) with the GH5 xylanase of SEQ ID NO: 4 at different enzyme concentrations compared to blank and the commercial GH11 xylanase Ronoxyme WX.

TABLE 4
Xylose release from DFDSM using GH5 xylanase SEQ ID NO: 18

| | | | % solu- | | |
|---|---|---|---|---|---|
| | Conc. | Soluble | bilised | Signifi- | Std. |
| GH5 Xylanase | [mg EP/kg] | xylose (%) | xylose¹ | cance² | Dev. |
| Blank | 0 | 0.001 | 0.0004 | C | 0.001 |
| Ronozyme WX | 25 | 0.053 | 0.04 | C | 0.009 |
| (GH11) | | | | | |
| SEQ ID NO: 6 | 10 | 0.934 | 6.4 | B | 0.154 |

¹Percentage solubilised xylose was calculated as described in example 3.
²ABC: Least squared values within a column not sharing a capital letter differ significantly (P < 0.05 all pairs Tukey-Kramer HSD).

Table 4 shows the amount of xylose measured after acid hydrolysis of supernatants (% of dry matter and % solubilized xylose of total xylose) when incubating defatted de-starched maize (DFDSM) with the GH5 xylanase of SEQ ID NO: 6 at different enzyme concentrations compared to blank and the commercial GH11 xylanase Ronoxyme WX.

As can be seen from tables 2, 3 and 4, the GH5 xylanases are significantly better at releasing xylose from defatted de-starched maize than either blank of the commercial GH11 xylanase Ronoxyme WX.

### Example 5

### Enzymatic Preparation of Corn Fiber Gum Hydrolysates

The purified enzymes used for the hydrolysis was SEQ ID NO: 2, SEQ ID NO: 4, SEQ ID NO: 6, SEQ ID NO: 7, SEQ ID NO: 8 (pentamodular GH5_34 from Clostridium thermocellum NzyTech (Catalogue Number (SKU): CR0061, Correia et al. J. Biol. Chem. 2011, 286:22510-22520)), Pentopan mono, Pulpzyme, and Shearzyme. For the commercial products the main xylanase was purified product by anion exchange chromatography and tested.

All reactions were performed with CFG1 at 3.1% DM as the final concentration. The assay also contained 45 mM MES buffer pH6 and the final enzyme concentrations tested were: 41, 18, 7.3, and 1.8 mg/L for 2 hours and 41 mg/L for 24 hrs at 40° C. for most enzymes. SEQ ID NO: 7 was assayed at 30° C. due to low thermostability. All reactions were stopped at 95° C. for 10 min and analyzed by HPLC-SEC on an ICS-3000 with RI detection (Dionex). The columns used were a PWXL guard column and a PWXL-3000 and -5000 (Tosoh) connected in series. The eluent was 50 mM NaOAc pH 5 and the flow rate 0.5 ml/min. retention times were compared to a pullulan molecular weight standard between 803-1.3 kDa and glucose, maltotriose and maltopentaose.

### Results

In the following tables the characteristics of the corn fiber gum and the respective products made by enzymatic treatment are given.

TABLE 5
Characteristics of the alkali solubilized corn fiber gum.

| Mp (Da) | Mn (Da) | Mw (Da) | Polydispersity |
|---|---|---|---|
| 227492 | 129701 | 302344 | 2.3 |

TABLE 6
Characteristics of corn fiber gum hydrolysates made using
the main GH11 xylanase activity in Pentopan Mono ™. The
calculations are based on the fraction above DP2.

| Enzyme | Incubation | | | | |
|---|---|---|---|---|---|
| conc. | time | | | | |
| (mg/L) | (hrs) | Mp (Da) | Mn (Da) | Mw (Da) | Polydispersity |
| 1.8 | 2 | 228839 | 124375 | 290617 | 2.34 |
| 7.3 | 2 | 184165 | 117007 | 263390 | 2.25 |
| 18 | 2 | 162696 | 117212 | 257179 | 2.19 |
| 41 | 2 | 205511 | 113496 | 254255 | 2.24 |
| 41 | 24 | 209217 | 115376 | 243574 | 2.11 |

TABLE 7
Characteristics of corn fiber gum hydrolysates made using
the main GH11 xylanase activity in Pulpzyme ™.
The calculations are based on the fraction above DP2.

| Enzyme | Incubation | | | | |
|---|---|---|---|---|---|
| conc. | time | | | | |
| (mg/L) | (hrs) | Mp (Da) | Mn (Da) | Mw (Da) | Polydispersity |
| 1.8 | 2 | 217344 | 126520 | 285321 | 2.26 |
| 7.3 | 2 | 207962 | 124297 | 265849 | 2.14 |
| 18 | 2 | 189005 | 117782 | 249998 | 2.12 |
| 41 | 2 | 193856 | 119805 | 251758 | 2.10 |
| 41 | 24 | 211270 | 115520 | 245405 | 2.12 |

TABLE 8
Characteristics of corn fiber gum hydrolysates made using
the main GH10 xylanase activity in Shearzyme ™
500 L. The calculations are based on the fraction above DP2.

| Enzyme | Incubation | | | | |
|---|---|---|---|---|---|
| conc. | time | | | | |
| (mg/L) | (hrs) | Mp (Da) | Mn (Da) | Mw (Da) | Polydispersity |
| 1.8 | 2 | 212907 | 105298 | 246478 | 2.34 |
| 7.3 | 2 | 208994 | 84282 | 221618 | 2.63 |
| 18 | 2 | 197932 | 74413 | 205479 | 2.76 |
| 41 | 2 | 194088 | 69845 | 197069 | 2.82 |
| 41 | 24 | 174418 | 44927 | 160978 | 3.58 |

TABLE 9
Characteristics of corn fiber gum hydrolysates made
using the family GH5_35 xylanase of SEQ ID NO: 2.
The calculations are based on the fraction above DP2.

| Enzyme | Incubation | | | | |
|---|---|---|---|---|---|
| conc. | time | | | | |
| (mg/L) | (hrs) | Mp (Da) | Mn (Da) | Mw (Da) | Polydispersity |
| 1.8 | 2 | 166060 | 20266 | 132629 | 6.54 |
| 7.3 | 2 | 63011 | 13211 | 70669 | 5.35 |
| 18 | 2 | 38557 | 9821 | 46983 | 4.78 |
| 41 | 2 | 21607 | 8057 | 35598 | 4.42 |
| 41 | 24 | 8701 | 6348 | 24976 | 3.92 |

TABLE 10
Characteristics of corn fiber gum hydrolysates made
using the family GH5_35 xylanase of SEQ ID NO: 4.
The calculations are based on the fraction above DP2.

| Enzyme | Incubation | | | | |
|---|---|---|---|---|---|
| conc. | time | | | | |
| (mg/L) | (hrs) | Mp (Da) | Mn (Da) | Mw (Da) | Polydispersity |
| 1.8 | 2 | 172666 | 22324 | 145242 | 6.51 |
| 7.3 | 2 | 63280 | 12591 | 70743 | 5.62 |
| 18 | 2 | 39090 | 9358 | 47688 | 5.1 |
| 41 | 2 | 20815 | 7618 | 35496 | 4.66 |
| 41 | 24 | 8837 | 5722 | 22324 | 3.9 |

TABLE 11
Characteristics of corn fiber gum hydrolysates made
using the family GH5_21 xylanase of SEQ ID NO: 6.
The calculations are based on the fraction above DP2.

| Enzyme | Incubation | | | | |
|---|---|---|---|---|---|
| conc. | time | | | | |
| (mg/L) | (hrs) | Mp (Da) | Mn (Da) | Mw (Da) | Polydispersity |
| 1.8 | 2 | 8228 | 8368 | 28884 | 3.45 |
| 7.3 | 2 | 4937 | 4709 | 14995 | 3.18 |
| 18 | 2 | 4735 | 4007 | 12885 | 3.22 |
| 41 | 2 | 2318 | 3645 | 10920 | 3.00 |
| 41 | 24 | 2268 | 2864 | 8559 | 2.99 |

TABLE 12
Characteristics of corn fiber gum hydrolysates made
using the family GH5_34 xylanase of SEQ ID NO: 7.
The calculations are based on the fraction above DP2.

| Enzyme | Incubation | | | | |
|---|---|---|---|---|---|
| conc. | time | | | | |
| (mg/L) | (hrs) | Mp (Da) | Mn (Da) | Mw (Da) | Polydispersity |
| 1.8 | 2 | 193478 | 74799 | 234258 | 3.13 |
| 7.3 | 2 | 82734 | 24762 | 137377 | 5.55 |
| 18 | 2 | 47096 | 14798 | 78416 | 5.30 |
| 41 | 2 | 16107 | 8499 | 41935 | 4.93 |
| 41 | 24 | 4541 | 4390 | 16530 | 3.77 |

TABLE 13
Characteristics of corn fiber gum hydrolysates made
using the family GH5_34 pentamodular arabinoxylanase
of SEQ ID NO: 8 (available from NzyTech). The calculations
are based on the fraction above DP2.

| Enzyme | Incubation | | | | |
|---|---|---|---|---|---|
| conc. | time | | | | |
| (mg/L) | (hrs) | Mp (Da) | Mn (Da) | Mw (Da) | Polydispersity |
| 1.8 | 2 | 137128 | 54827 | 229844 | 4.19 |
| 7.3 | 2 | 77794 | 31050 | 153021 | 4.93 |
| 18 | 2 | 38688 | 11646 | 83398 | 7.16 |
| 41 | 2 | 8894 | 7192 | 38017 | 5.29 |
| 41 | 24 | 2533 | 2703 | 10810 | 4.00 |

The corn fiber gum hydrolysate data clearly showed that all the tested GH5 xylanases from subfamilies GH5_21, GH5_34 and GH5_35 have the capacity to degrade this highly substituted xylan. The DP1-DP2 fraction was below 1% under all tested conditions.

The two family GH11 xylanases tested did not degrade the xylan at all while the GH10 xylanase present in Shearzyme 500 L only had moderate corn fiber gum-degrading capacity.

Based on the above results the effects of adding GH5 xylanases in a starch to ethanol process was tested.

### Example 6

### Ethanol Yield Improvement Using a GH5_35 and GH5_21 Xylanase Added in Saccharification/Fermentation

Liquefaction of a mash from milled corn was performed at 85° C. using a commercial alpha amylase, Liquozyme™ SODS (available from Novozymes NS) according to the table below.

| | Enzyme(s)/kg/ | Temp. | Time | | | Mixing | Calcium |
|---|---|---|---|---|---|---|---|
| Mash | t DS | ° C. | Min. | DS % | pH | Rpm | ppm |
| | Corn: 0.3 kg | 85 | 120 | 28 | 5.5 | 80 after | 30 |
| | SCDS/t DS | | | | | 20 min at | |
| | | | | | | 300 | |

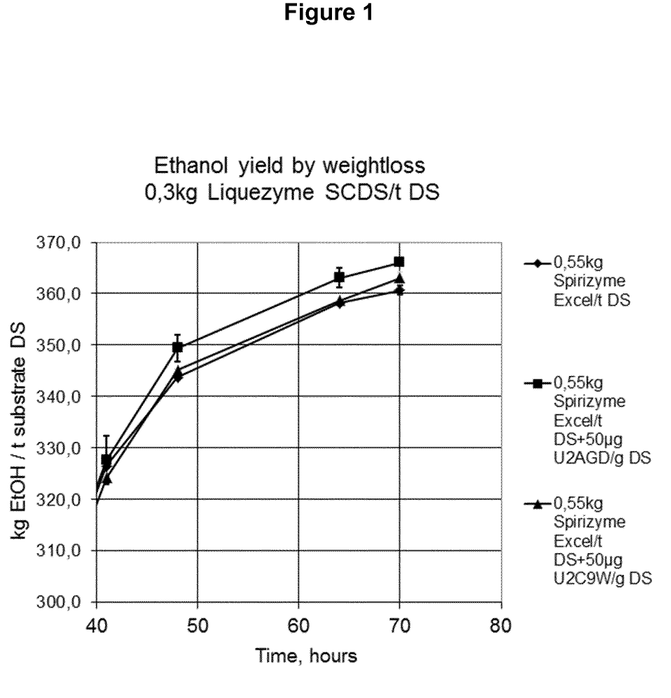

The liquefied mash was split into three parts and simultaneous saccharification and fermentation performed according to the below table. A commercial glucoamylase product, Spirizyme Excel™ (available from Novozymes NS) was used for saccharification and the yeast was Ethanol Red™ (available from Lallemand). Fermentation was followed by weight loss measured twice a day and by HPLC at the end of fermentation. The control sample only contained the glucoamylase enzymes. In addition one sample contained glucoamylase and the GH5_35 xylanase (U2AGD/SEQ ID NO: 4) and another sample contained glucoamylase and the GH5_21 xylanase (U2C9W/SEQ ID NO: 6). The ethanol yield is shown in FIG. 1 and demonstrates that the addition of a GH5 xylanase increased the ethanol yield compared to the control. In particular the GH5_35 xylanase resulted in an increase of around 2%.

| | | | Time | | Yeast |
|---|---|---|---|---|---|
| Sample | Enzyme(s) | T (° C.) | (Hours) | pH | (mill/ml) |
| Control | 0.55 kg Spirizyme | 32 | 96 | 4.5 | 30 |
| | Excel/t DS | | | | |
| GH5_35 | 0.55 kg Spirizyme | 32 | 96 | 4.5 | 30 |
| | Excel/t DS + 50 μg | | | | |
| | SEQ ID NO: 4/g DS | | | | |
| GH5_21 | 0.55 kg Spirizyme | 32 | 96 | 4.5 | 30 |
| | Excel/t DS + 50 μg | | | | |
| | SEQ ID NO: 6/g DS | | | | |

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1. A process for producing fermentation products from starch-containing material comprising the steps of:
a) liquefying starch-containing material using an alpha-amylase:
b) saccharifying the liquefied material using a glucoamylase;
c) fermenting using a fermenting organism, wherein saccharification is carried out in the presence of a GH5 xylanase.

2. The process according to claim 1, wherein the starch containing material comprises maize, corn, wheat, rye, barley, triticale, sorghum, switchgrass, millet, pearl millet, foxtail millet or in a processed form such as milled corn, milled wheat, milled rye, milled barley, milled triticale, milled maize, defatted maize, defatted destarched maize, milled sorghum, milled switchgrass, milled millet, milled foxtail millet, milled pearl millet.

3. The process according to claim 1, wherein the starch-containing material comprises highly branched xylan.

4. The process according to claim 1, wherein the GH5 xylanase is selected from subfamily 21, 34, or 35.

5. The process according to claim 4, wherein the subfamily 21 GH5 xylanase is selected from the xylanases shown as amino acids 36 to 633 of SEQ ID NO: 6, or a GH5 xylanase having at least 75% identity to amino acids 36 to 633 of SEQ ID NO: 6.

6. The process according to claim 4, wherein the subfamily 34 GH5 xylanase is selected from the xylanases shown as SEQ ID NO: 7, SEQ ID NO: 8, or a xylanase having at least 75% identity to SEQ ID NO: 7, or SEQ ID NO: 8.

7. The process according to claim 4, wherein the subfamily 35 GH5 xylanase is selected from the xylanases shown as amino acids 37 to 573 of SEQ ID NO: 2, amino acids 36 to 582 of SEQ ID NO: 4, or a xylanase having at least 75% identity to amino acids 37 to 573 of SEQ ID NO: 2, or amino acids 36 to 582 of SEQ ID NO: 4.

8. The process according to claim 1, wherein the plant material comprises corn, sorghum, wheat, rye, barley, or triticale.

9. The process according to any claim 1, wherein the fermentation product is ethanol.

10. The process according to claim 1, wherein the fermenting organism is yeast.

11. The process according to claim 1, wherein steps b) and c) are performed simultaneously.

12. A method for producing ethanol from starch containing material, comprising saccharifyinq a liquefied starch containing material using a GH5 xylanase.

13. The method according to claim 12, wherein the GH5 xylanase is selected from subfamily 21, 34, or 35.

14. The method according to claim 12, wherein starch containing material comprises corn, sorghum, wheat, rye, barley, or triticale.

15. The process according to claim 10, wherein the fermenting organism is Saccharomyces sp.

16. The process according to claim 10, wherein the fermenting organism is Saccharomyces cerevisiae.


SEQUENCE LISTING (8 sequences)

SEQ ID NO: 1 - DNA, 1722 bp, Artificial
  Codon optimized gene encoding a GH5_35 xylanase from Paenibacillus illinoisensis
atgaagaaaccgttggggaaaattgtcgcaagcaccgcactactcatttctgttgcttttagttcatcgatcgcatcggctcatcagcaccaacaccagcatcctaggtgggttggcatgccgatgggcaaactgcatgtttcaggcaaaaatctggtcaatagcaataatcaaccggttctgctgaatggctggcatcaaccgtcaggcgcatattggacatatcaatcatcaaactattatctgaatctgcatggaaataatcgccatgcggcaacactggcatatctgaaagatatcacagatacatttgcagacacgtcaccgaaatatggctcaaatcatggctggaatatgaatcaggtccgcctgtttattgatcgcgaagatatgggagatgttgcagcaggcacatataactttgcaggcgttcaaacagttacacagaatgtcattattccgtatatccagtatgcgaaaacgaaaggcgtttatgttgttctgggcctggattttacactgaaagatgatcaagcaacaacagcgagcaatctgcagaaatttaaccaaatttggggctatctggcatcaagaccggaaatcaaatcagcagataacgtccattttgaactgattaacgaaccggttaaaagctatgcaaatggccattggggaggctataatggcgaaaatgattttgtcgatcattggaatgatctgcgcaattttcagaacagcattatttcaacaattcgctcacaaggcgcagataatgttatttgggcagcaggcctgggctataatcagttttattcactgacagcatcacatccgctgacagatccgctgaataactatggctatgcagttcattggtatccgggatatggcgcatatgataactttagcattctgcaggatcagtggaacacaaatgttaaagcagcagcagataaatatccgattaacatcacggaagtcacatggtttaaaaacaaacctggcgattcagcatactggaatctttttaatggcagcaatgaaggctttggcacaaacacaaaaacgatctttaatgcagcaggcaatgttagcattgcagcacatatgaatggctttattctggaaccgggacaaagatcatcatttgcagatccgacagcaggcctgaaatgggatggcgacgcatcaagatcagcaatgggcagatttctgtttaattggtatcatgaaagagcgcaatcatatcctggcggaaatggcggaggaccgacaacaggcctggtttcaggcgcaacatacaaaattgttgcacgccatagcaacaaagtcattgatgtccctggcggacaaaatcaaaacaatctgcaactgcaacagtggtcagatctgggaggcaatccgcaaaaatgggttctgacatcaattggcggaggctcatatacactgacatcagttaattcaccggacaaagtgattgatattcgcaatggcacactgacaaatggcgaagcagttcaactgatgtcaaatctgaatacaacagcgcagcattttaaagtcaatgatctgggcaatggctattggagcattattaacgtcaacagcaacaaagcgatcgaagttgaaaatgcatcaacatcagatggcgcaaaactgcagcaaaatacatatacaggcgcaacaaatcagcagtggaaatttatcgcagtcagcaattaa

SEQ ID NO: 2 - protein, 573 aa, Artificial
  GH5_35 xylanase from Paenibacillus illinoisensis including a signal peptide from Bacillus clausii and a His-tag
MKKPLGKIVASTALLISVAFSSSIASAHQHQHQHPRWVGMPMGKLHVSGKNLVNSNNQPVLLNGWHQPSGAYWTYQSSNYYLNLHGNNRHAATLAYLKDITDTFADTSPKYGSNHGWNMNQVRLFIDREDMGDVAAGTYNFAGVQTVTQNVIIPYIQYAKTKGVYVVLGLDFTLKDDQATTASNLQKFNQIWGYLASRPEIKSADNVHFELINEPVKSYANGHWGGYNGENDFVDHWNDLRNFQNSIISTIRSQGADNVIWAAGLGYNQFYSLTASHPLTDPLNNYGYAVHWYPGYGAYDNFSILQDQWNTNVKAAADKYPINITEVTWFKNKPGDSAYWNLFNGSNEGFGTNTKTIFNAAGNVSIAAHMNGFILEPGQRSSFADPTAGLKWDGDASRSAMGRFLFNWYHERAQSYPGGNGGGPTTGLVSGATYKIVARHSNKVIDVPGGQNQNNLQLQQWSDLGGNPQKWVLTSIGGGSYTLTSVNSPDKVIDIRNGTLTNGEAVQLMSNLNTTAQHFKVNDLGNGYWSIINVNSNKAIEVENASTSDGAKLQQNTYTGATNQQWKFIAVSN

SEQ ID NO: 3 - DNA, 1749 bp, Artificial
  Codon optimized gene encoding a GH5_35 xylanase from Paenibacillus sp 18054
atgaagaaaccgttggggaaaattgtcgcaagcaccgcactactcatttctgttgcttttagttcatcgatagcatcagcacatcatcatcaccatcatcctaggttgacggttccgcccggcgccccggccgaggcttggtctggcatgcctacgcccaagcttcatgtcagcggcaaccaactggtaaatgcaaacggacagcctgtcctgctaagcgggtggcatcagccttccgggtcctactggacgtatcagagcagcagttactacctggaccgtaacggcggaaaccggcatgccgccaacctggcgtatctcaaggatatcacagacacctttacggacacctcgcccaaatacgggaacaaccacggctggtatatgaatcaggttcggctattcattgatcgcgaggatatgggggatgtcgccgaaggcacttacaactttgctggattgcaggcggttacgcaaaatgtgattattccgtacattaattatgcgagaacaaaagggctttatgtgacgctcggactggattttacgctcaaagacaatcaggcgaccacacaggccaatttggacaagttcaatcagatttggagttatctagcatccagaccggaaataagaagcgctgacaacgtcatgtttgaaattattaacgagccggtattgtcgtatgcggacggcagatggggcggtcatccgtccgaccctcattttatagcattctggaacgatttgcgcagttttcaaaactctatcatctcctccattcgcgcacaaggagcggataatgtgatctgggcggccgggctcggctgggatcagtactaccagttgtgtgcgtcgcatcctctgacggacccgctcaataatgtaggttatgcggttcactggtatccgggatatggagcaggggataactattcggtgcttcagcaacaatgggatacgaatattaagccatgcgccgacaactatccgatcaatataacggaaacgacctggttcaagcgactgcctggcgattcggattactggaatttgttcaatggctcgagcgagggcttcggcaaaaatacgaaagcaatctttactgcggcgggcaatgccagcattgccgtccatatgaacggctttttattggcgccgggagcaagaagctcatttgccgatccgactgccggtctgttatacgacggaaatacagctcgagacggcatggcccgctttatattcgagtggtattacgaaagagcgcagttcttgccgtggaatggcatttggaacggactgttcacaggatcgacctacaagttcgtgaatcgggcaaccggcaaaaatatggatgtgccgggcggacaaaacaataataatctgcaattgaaccaatggacggataatggagcaacggcgcagcgctgggtcgtagacgatatgggcactttcaacaatatatatcgcatgaagagcgtcagctcatcggacggcaaggtaatggatgtccgcaacggcaccaaaaacaatggagaagccattcagcttatgcaggacttctcgaacacggcgcagcgtttccggattattcgacttagcaacggctattggagcattattaatgtcaacagcaacaaagcggttgaggtcgccggcggcgcttctcatgacggagcgctattgcaacagaacatgtatcgcggagatcatcatcaacagtggcagctggttcagattcaataa

SEQ ID NO: 4 - protein, 582 aa, Artificial
  GH5_35 xylanase from Paenibacillus sp. including a signal peptide from Bacillus clausii and a His-tag
MKKPLGKIVASTALLISVAFSSSIASAHHHHHHPRLTVPPGAPAEAWSGMPTPKLHVSGNQLVNANGQPVLLSGWHQPSGSYWTYQSSSYYLDRNGGNRHAANLAYLKDITDTFTDTSPKYGNNHGWYMNQVRLFIDREDMGDVAEGTYNFAGLQAVTQNVIIPYINYARTKGLYVTLGLDFTLKDNQATTQANLDKFNQIWSYLASRPEIRSADNVMFEIINEPVLSYADGRWGGHPSDPHFIAFWNDLRSFQNSIISSIRAQGADNVIWAAGLGWDQYYQLCASHPLTDPLNNVGYAVHWYPGYGAGDNYSVLQQQWDTNIKPCADNYPINITETTWFKRLPGDSDYWNLFNGSSEGFGKNTKAIFTAAGNASIAVHMNGFLLAPGARSSFADPTAGLLYDGNTARDGMARFIFEWYYERAQFLPWNGIWNGLFTGSTYKFVNRATGKNMDVPGGQNNNNLQLNQWTDNGATAQRWVVDDMGTFNNIYRMKSVSSSDGKVMDVRNGTKNNGEAIQLMQDFSNTAQRFRIIRLSNGYWSIINVNSNKAVEVAGGASHDGALLQQNMYRGDHHQQWQLVQIQ

SEQ ID NO: 5 - DNA, 1902 bp, Artificial
  Codon optimized gene encoding GH5_21 xylanase from unknown organism
atgaagaaaccgttggggaaaattgtcgcaagcaccgcactactcatttctgttgcttttagttcatcgatagcatcagcacatcatcatcaccatcatcctaggtggcgtggcatgagaatgccggaactgtttatcaaaggcagatatctgatggcgaaagatatgaatggcaacgatagcattgttaatctgcatggctttggccaaacatatagcgcgtattttaacggctatgcgtggtgcaaaaatccggatggctcagttaattggggcaaaacaaaagatgcagcagcatgcgttaaatggaataaagaacaaattggcctgatgctggatcatggctggaaagttaattggctgagactgcatatggatccggcatggtcaaataatgaaacaaaagtcaatcaatggcagagccaacatccgggaacatattattcagaaaatctgatcgtcgcgtttgatatgaacctgtttaaaaaatatctggatgaaatctttattccgatggcggaatatgcgattgaaaacggcatttatgttgttatgcgtccgcctggcgtttgtccgcaaaaactgacagttggagatgaatatcagcagtacctgattaaagtctggacatatgtttgcagccatgaaaaactgaaaaacaatccgtatattatgtttgaactggcgaacgaaccgatcgatatgaatgatggcaatggcaattatacgtcatggtcagatggctcacagaaaaactgcacgaaattttttcagaaaattgtcgacgaaattagagcagtcggctgcaataacattctgtgggttccgggactggcatatcaacaaaattatcaaggctatgtcaaatacccgattgtcggcgaaaatattggctttgcagttcattgctatccgggatggtatggctcagattcagaagttgcatcagcagaacaacaaattgtcacaaacggcaatacgtatgcggattttcaatcaggctggtcagcaagcattgatggcgtttcaaaacttagaccgattatcgtcacagaaatggattgggcaccgaaaaaatacaattcatcatggggcaaagcaacgacaggcaaactgggaggcgttggctttggcaataactttaaatacatcatggacaaaacaggcaacgttagctggatgctgtttacagatgcagataaactggcgaaatatgatgattcaaaagcagatggcagcacgtttctgacagatccggaagcatgccctagaccggtttatagatggtataaagaatatgcagaaccgggatggaaatttgttgaaacactggcagatgaattttacatgtttccgggaacaaacagcatttttagcccgaacatttgggaaaaaggcacactgacaaaaaatgatgatggctcaagaacactggtcacaggccaatatggctttggaggctggaaatttggcggaggcctggatatgtcaggctataaatacctggttctgaacctgacaaaagcaccggcatcaaatcaatggtcactgagactgtttgatgtcgataactattggacagacccgtatatgaaagatgtcaaatcaagcacaagagtcgttgtcgatctgcagaatatgaaaaatagcaaaggcgttaaagtcgacccgagccatatctatattctgggcctgtggtcaacaggcggaacaccgattacaattaaagatatctatctgacaaataatagcgattattcaccggaatcaacaggcatttcagaaacacttgcagaaaaaagactggacacaccgatttataacctgtcaggccaaagagttacagaaccgagaaatggccatgtctatattcgcaacggcaaaaaattcatttacaaataa

SEQ ID NO: 6 - protein, 633 aa, Artificial
  GH5_21 xylanase including a signal peptide from Bacillus clausii and a His tag
MKKPLGKIVASTALLISVAFSSSIASAHHHHHHPRWRGMRMPELFIKGRYLMAKDMNGNDSIVNLHGFGQTYSAYFNGYAWCKNPDGSVNWGKTKDAAACVKWNKEQIGLMLDHGWKVNWLRLHMDPAWSNNETKVNQWQSQHPGTYYSENLIVAFDMNLFKKYLDEIFIPMAEYAIENGIYVVMRPPGVCPQKLTVGDEYQQYLIKVWTYVCSHEKLKNNPYIMFELANEPIDMNDGNGNYTSWSDGSQKNCTKFFQKIVDEIRAVGCNNILWVPGLAYQQNYQGYVKYPIVGENIGFAVHCYPGWYGSDSEVASAEQQIVTNGNTYADFQSGWSASIDGVSKLRPIIVTEMDWAPKKYNSSWGKATTGKLGGVGFGNNFKYIMDKTGNVSWMLFTDADKLAKYDDSKADGSTFLTDPEACPRPVYRWYKEYAEPGWKFVETLADEFYMFPGTNSIFSPNIWEKGTLTKNDDGSRTLVTGQYGFGGWKFGGGLDMSGYKYLVLNLTKAPASNQWSLRLFDVDNYWTDPYMKDVKSSTRVVVDLQNMKNSKGVKVDPSHIYILGLWSTGGTPITIKDIYLTNNSDYSPESTGISETLAEKRLDTPIYNLSGQRVTEPRNGHVYIRNGKKFIYK

SEQ ID NO: 7 - protein, 620 aa, Acetivibrio cellulyticus
ADPQRGRPYLNSARTTFVGDNGQPLRGPYISTEWTSAAPYDQIARIKNLGFNAVHHYAECFDINYPNAGSKSPGYAATEIDKVVERTRELGLYLVMTIGNGANNGNHNTRYAKDFWSFYSSRYANETHVLYEIHNEPVAWGPPYSSTTATPTGAVEMNVDVYKTIRANAPKTPVLIFSYSVFGGTGGTTEALKDIQAFNSAVFGKQDAVWTNEAVAFHGYAGWEATSTAVDGLLKAGYPCFMTEYAGGAWGSGTGGFDIQLTSELERMGVSWLTFQYIPPSGVSDDVTKPEYFSALVENAGLSWKPDYGNWPAARGVHGNGGLPRKTSTWVNNFLTGTTRIEAEDFDWGGNDVSFYDKDSENKGAQYRLDEAVDIETTKDADGGYNVGWIEDGEWLEYTIWVQHPGYYNLALRVANNSGGSVQVNFGNQDKTGTWVLPVTGGVQTWKTDTRQVFLGSGRQKLRINALSGGFNLNWIELSPVSTGPIADGTYKFLNRANTMTLQEVTSNNSIVTSTYKGTADQHWKIQHIGGGQYRISSAGRGWNWNWWMGFGTVGWWGTGSGTCFIIRPTGDGYYRFVLVNDGTNLEISNNDSSKIEGKAYHEGANQQWAIQLPSAPVFP

SEQ ID NO: 8 - protein, 865 aa, Unknown
  Commercial GH5_34 xylanase from NZYtech
AIALSILSFIPNRAYASPQRGRPRLNAARTTFVGDNGQPLRGPYTSTEWTAAAPYDQIARVKELGFNAVHLYAECFDPRYPAPGSKAPGYAVNEIDKIVERTRELGLYLVITIGNGANNGNHNAQWARDFWKFYAPRYAKETHVLYEIHNEPVAWGPPYSSSTANPPGAVDMEIDVYRIIRTYAPETPVLLFSYAVFGGKGGAAEALKDIRAFNKAVFGNENAVWTNEAVAFHGYAGWQETTIAVEELLKAGYPCFMTEYAGGAWGSGMGGLDVELTYELERLGVSWLTFQYIPPTGVSDDVTKPEYFSALVENSGLSWTPDYGNWPAARGVYGNGGLARETATWINNFLTGTTRIEAEDFDWGGNGVSYYDTDSVNVGGQYRPDEGVDIEKTSDTGGGYNVGWISEGEWLEYTIRVRNPGYYNLSLRVAGISGSRVQVSFGNQDKTGVWELPATGGFQTWTTATRQVFLGAGLQKLRINALSGGFNLNWIELSPISTGTIPDGTYKFLNRANGKTLQEVTGNNSIITADYKGITEQHWKIQHIGGGQYRISSAGRGWNWNWWMGFGTVGWWGTGSSTCFIISPTGDGYYRIVLVGDGTNLQISSGDPSKIEGKAFHGGANQQWAILPVSAPAFPTGLSAVLDSSGNTANLTWNAAPGANSYNVKRSTKSGGPYTTIATNITSTNYTDTGVATGTKYYYVVSAVSNGVETLNSAEAILQYPKLTGTVIGTQGSWNNIGNTIHKAFDGDLNTFFDGPTANGCWLGLDFGEGVRNVITQIKFCPRSGYEQRMIGGIFQGANKEDFSDAVTLFTITSLPGSGTLTSVDVDNPTGFRYVRYLSPDGSNGNIAELQFFGTPAGEENDDVH